(19) United States
(12) Patent Application Publication
(10) Pub. No.: US 2026/0020918 A1
(43) Pub. Date: Jan. 22, 2026

(54) SYSTEMS AND METHODS FOR DATA GATHERING AND PROCESSING

(71) Applicant: PABBAN DEVELOPMENT, INC., IRVINE, CA (US)

(72) Inventors: JACOB HERBERT, Irvine, CA (US); BRADFORD H. HACK, Irvine, CA (US); JOSE MAURICIO GARCIA PRIETO, Irvine, CA (US)

(21) Appl. No.: 18/993,514

(22) PCT Filed: Jul. 11, 2023
(86) PCT No.: PCT/US2023/069915

§ 371 Date: Jan. 10, 2025

Related U.S. Application Data
(60) Provisional application No. 63/388,022, filed on Jul. 11, 2022.
(60) Provisional application No. 63/398,330, filed on Aug. 16, 2022.

(51) Int. Cl. A61B 34/20; A61B 90/00; A61B 90/50; G16H 40/63

(52) U.S. Cl. CPC A61B 34/20; G16H 40/63; A61B 2034/2072; A61B 2090/372; A61B 2090/502

(57) ABSTRACT

A system for improving efficiency in a medical facility includes one or more sensors configured to wirelessly obtain information from a medical procedure area and output a first signal related to the obtained information, a processor configured to survey the first signal and identify one or more descriptors in the signal, and an output configured to describe one or more changes related to operation of the medical procedure area based at least in part on the one or more descriptors.

### BACKGROUND OF THE INVENTION

### Field of the Invention

The field of the invention generally relates to computer-aided planning, simulation or modelling of surgical operations, including, but not limited to identification means for patients or instruments. The field of the invention further relates to facial shields for use in medical procedures, including their use with heads-up displays.

### SUMMARY OF THE INVENTION

In a first embodiment of the present disclosure, a system for improving efficiency in a medical facility includes one or more sensors configured to wirelessly obtain information from a medical procedure area and output a first signal related to the obtained information, a processor configured to survey the first signal and identify one or more descriptors in the signal, and an output configured to describe one or more changes related to operation of the medical procedure area based at least in part on the one or more descriptors.

In another embodiment of the present disclosure, a system for aiding a medical procedure includes a cover configured to protect the head of a user from contaminants in an ambient environment external to the cover, the cover comprising a substantially transparent facial shield, a projector configured to project an image on at least a portion of the facial shield that is configured to reside within a field of vision of the user, a sensor configured to wirelessly obtain information from a medical implant within a medical procedure area and output a first signal related to the obtained information, and a processor configured to determine an identity characteristic of the implant based on the first signal.

### BRIEF DESCRIPTION OF THE DRAWINGS

FIG. 1 is a perspective view of an operating room utilizing a system for data gathering and processing, according to an embodiment of the present disclosure.

FIG. 2 is a flow chart illustrating using the speech of medical personnel to optimize a procedure.

FIG. 3 is perspective view of a system for aiding a medical procedure, according to an embodiment of the present disclosure.

FIG. 4 is a plan view of portions of a surgical kit of FIG. 3.

FIG. 5 is a perspective view of the system for aiding a medical procedure in video acquisition selection mode.

FIG. 6 is an elevation view of an exemplary image provided to a user, according to an embodiment of the present disclosure.

FIG. 7 is an elevation view of an overall implant image provided to the user, according to an embodiment of the present disclosure.

FIG. 8 is a perspective view of the system for aiding a medical procedure in electromagnetic acquisition selection mode.

### DETAILED DESCRIPTION OF THE ILLUSTRATED EMBODIMENTS

The disclosure generally relates to data gathering and data processing in surgical or other medical procedures. The data gathering and data processing can include eavesdropping by one or more sensors, including, but not limited to, one or more visual sensor, one or more audio sensor, one or more vibration sensor, one or more location sensor, one or more chemical sensor (sniffer), or one or more heat sensor. In some embodiments, data gathering capability can be incorporated into displays for use with facial covers or facial shields, including those used in personal protection systems, including, but not limited to personal environmental protections systems. The data gathering and data processing can be used to aid in the optimization of medical procedures, including surgeries. The personal protection systems often include a headgear structure which is worn by an individual to protect from particulate material. In some embodiments, the personal protection systems can provide filtered air to the user. The disclosure also relates to devices, apparatus or methods for life-saving, including devices for medical use. The disclosure also relates to respirators or can relate to respiratory apparatus, such as respiratory apparatus for medical purposes, including apparatus with filter elements. The disclosure also describes information systems that couple information related to the performance and optimization of a medical procedure with a facial shield. The facial shield can in some embodiment be provided as a heads-up display to be utilized by a user.

There are several types of air flow, filtration and protective systems which are known in the art. Several types of such systems are currently available on the market for use in surgical arenas, in “clean room” environments, or in hazardous/contaminated environments.

Some of the existing systems include hoods, gowns, filters, and the like. In some instances, the air filters are built into the helmet structure. Known units frequently include external sources of air such as gas cylinders, air lines or the like which are connected to the helmet structure by tubes, hoses or the like. Other systems do not have hoses, such as no hose respirators and no hose powered air purifying respirators.

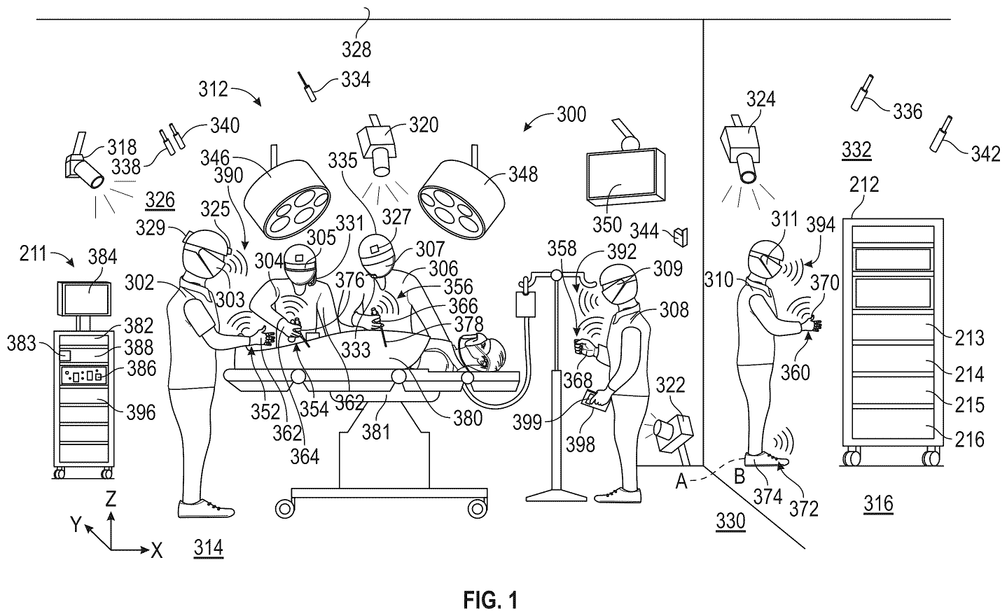

FIG. 1 illustrates an operating room 300 in which medical personnel 302, 304, 306, 308, 310 work to perform a surgery or other medical procedure. “Operating room” should be interpreted in a general sense, for the procedure that occurs need not be a surgical procedure. Other terms can be used for the operating room, or medical procedural space, such as catheter laboratory or cath lab, delivery room, operating theater, outpatient procedure room, or simply procedure room. The operating room 300 is shown in FIG. 1 having a first light 346 and a second light 348, and a display monitor 350. There are several general types of “procedure,” in terms of the level of definition. A procedure can be fully designed or substantially fully designed, meaning that substantially all steps are described in advance or at least well-known. In this case, the tasks of each of the medical personnel 302, 304, 306, 308, 310 occur in a particular order. The opposite extreme would be, for example, an emergency medical procedure for a patient that has been injured or otherwise afflicted by a rare or unknown occurrence, cause, or disease. In this case, the medical personnel 302, 304, 306, 308, 310 will have to improvise some or much of the procedure, albeit with the aid of their knowledge, habits, and/or muscle memory from prior training, skills, and experience. Many procedures tend to be in between the two extremes of designed and improvised. For example, many procedures follow a general structure comprising a series of steps to be completed, some or all in a particular order. However, certain occurrences during the procedure, or certain pieces of information that can only be learned during a procedure, can comprise additional inputs that require a variation in the procedure. The variation can involve the addition of one or more steps, a variation in one or more of the steps, a variation in the order of the steps, a removal of one or more of the steps, a change in the emphasis of one of more of the steps, a change in the particular personnel who perform one of more of the steps, the need for additional personnel to perform one of more of the steps, the equipment used or to be used, or other factors.

In general, whether a particular medical procedure is generally defined, generally improvised, or a mixture of the two, the personnel performing the procedure and the institution in which the procedure is performed can benefit from learning characteristics related to the procedure, in order to make future procedures more efficient, cost-effective, safe, rapid, quality-focused, efficacious, and/or repeatable. However, as medical personnel are performing a procedure, they are usually focused on the procedure itself, and most of all, the safety of the patient, and so often have to rely on their memory for much of the continuous improvement input. Embodiments for systems and methods for data gathering and processing disclosed herein allow the operating room 300 itself to continuously improve, and each of one or more procedures performed regularly in the institution to continuously improve, evolve, and become more efficient.

A system for data gathering and processing 312 is illustrated in FIG. 1 as set up within an operating room 300 comprising a main room 314 and a side room 316. A main control console 211 is configured to communicate (wirelessly or wired) with other sensor elements such as cameras, or other types of sensors (microphones, receivers, transmitters, transceivers, RFID readers that read RFID chips on objects, Global Positioning Satellite-GPS sensors, etc.). A first camera 318, second camera 320, and third camera 322 are secured within the main room 314, and a fourth camera 324 is secured within the side room 316. The first camera 318, second camera 320, and fourth camera 324 are suspended from the wall 326 of the main room 314, wall 332 of the side room 316, or ceiling 328 of the main room 314 or side room 316 of the operating room 300, and the third camera 322 is suspended from the wall 326 or floor 330 of the main room 314. Each camera 318, 320, 322, 324 acts as a visual sensor, recording data during a procedure. The types of recordable visual data that can be obtained by one or more of the cameras 318, 320, 322, 324 are listed below in Table 1. Cartesian, cylindrical, and/or spherical coordinate systems can be utilized. Lagrangian or Eulerian frames or reference can be utilized. Data other than that listed in Table 1 can also be recorded by one or more of the cameras 318, 320, 322, 324. In other embodiments, a camera 325 (e.g., carried on personnel 302) or camera 327 (e.g., carried on personnel 306) can be mounted on a respirator 329, 335 such as a powered air purifying respirator (PAPR) or other personal protective equipment (PPE).

TABLE 1

| Object Recorded | Type of Data Recorded |
|---|---|
| by Camera | and/or Calculated |
| Medical Personnel | Location (x, y, z) |
| Medical Personnel | Movement (velocity vector) v(x, y, z) |
| Medical Personnel | Movement (acceleration vector) a(x, y, z) |
| Patient | Location (x, y, z) |
| Patient | Orientation/Angulation (α, ⊖, φ) |
| Medical Equipment/Furniture | Location (x, y, z) |
| Medical Equipment/Furniture | Movement (velocity vector) v(x, y, z) |
| Medical Equipment/Furniture | Movement (acceleration vector) a(x, y, z) |
| Medical Instrument | Location (x, y, z) |
| Medical Instrument | Movement (velocity vector) v(x, y, z) |
| Medical Instrument | Movement (acceleration vector) a(x, y, z) |
| Medical Implant | Location (x, y, z) |
| Medical Implant | Movement (velocity vector) v(x, y, z) |
| Medical Implant | Movement (acceleration vector) a(x, y, z) |
| Hand of Medical Personnel | Location (x, y, z) |
| Hand of Medical Personnel | Movement (velocity vector) v(x, y, z) |
| Hand of Medical Personnel | Movement (acceleration vector) a(x, y, z) |
| Foot of Medical Personnel | Location (x, y, z) |
| Foot of Medical Personnel | Movement (velocity vector) v(x, y, z) |
| Foot of Medical Personnel | Movement (acceleration vector) a(x, y, z) |

Besides the cameras 318, 320, 322, 324, other types of sensors can alternatively or additionally be used to obtain data, for example: one or more microphone 334, 336, one or more transmitter 338, one or more receiver 340, or one or more transceiver 342. In some embodiments, the sensor can comprise a GPS (global positioning satellite) sensor 344. Any of the sensors or sensor components 318, 320, 322, 324, 334, 336, 338, 340, 342, 344 can be configured to determine location, orientation, or movement information (e.g., velocity or acceleration) of any “thing” that is in or near the operating room 300, or otherwise within the range of the sensor or sensor component 318, 320, 322, 324, 334, 336, 338, 340, 342, 344. Thus, very detailed, continuous or substantially continuous (e.g., continual) information can be captured over time of the personnel 302, 304, 306, 308, 310 or any item within the operating room 300 (or near the operating groom 300). In some cases, the movement of the personnel 302, 304, 306, 308, 310 can comprise movement of the entirety of a person, such as the movement of medical personnel 310 from point A to point B. In this particular case, point A is on main room 314 and point B is in side room 316, but in other cases, point A and point B may each be in the same room. In other cases, the movement of the personnel 302, 304, 306, 308, 310 may comprise the movement 352, 354, 356, 358, 360 of a hand 362, 364, 366, 368, 370. In other cases, the personnel 302, 304, 306, 308, 310 can comprise the movement 372 of a foot 374. In some cases, the movement 352, 354, 356, 358, 360 of a hand 362, 364, 366, 368, 370 can indicate the operation of an instrument 376 or the movement of an implant 378 being moved. For example, the implant 378 may be in the process of being implanted, removed, moved, or adjusted, in location with respect to a patient 380 (who may be in a prone, supine, lateral, Trendelenburg, or reverse Trendelenburg position or other positions). Or, the implant 378 may be in the process of being implanted, removed, moved, or adjusted, in location or with respect to itself (e.g., lengthened, shortened, tightened, loosened, angulated, activated, etc.). Or, the instrument 376 may be in the process of being inserted, retracted, or adjusted, either in location with respect to the patient 380 (e.g., prone, supine, lateral, Trendelenburg, reverse Trendelenburg, etc.), or with respect to itself (e.g., lengthened, shortened, tightened, loosened, angulated, activated, etc.) Additionally, the mere existence of an instrument 376 or implant 378, or the lack of the instrument 376 or implant 378 can be what is measured or communicated by the system 312. Alternatively, the unpackaging of an instrument 376, implant 378 or other product can be what is measured or communicated by the system 312. Furthermore, a microphone 331 or microphone 333 can be mounted on a respirator 329, 335, such as a powered air purifying respirator (PAPR) or other personal protective equipment (PPE). The cameras 325, 327 and microphones 331, 333 are configured to obtain additional information, or more of the same information as the other cameras and microphones.

If it is desired, the cameras 318, 320, 322, 325, 327 are able to determine location and movement of an object by buy utilizing and/or quantifying changes in sensed focal length, or by being configured to focus on a particular element (spot, mark) of the object. Three cameras 318, 320, 322 can even be set up to provide visual triangulation, which can then be utilized to calculate locations, movements, and/or distances of objects or spots or marks on the objects within view. Other sensors such as transmitters 338, receivers 340, transceivers 342, GPS sensors 344 can also be configured to provide location and movement of an object by sensing changes in measured signals from the objects, either reflected signals, or signals emanating from the object. Microphones 334, 336, 331, 333 can even be used to determine location or movement of objects. For example, the object can comprise a sound producer (piezoelectric, vibrator, loudspeaker, bell, clicker, alarm, etc.) configured to produce a characteristic sound (ring, hum, buzz, click, chirp) that can be sensed by the one or more microphones 334, 336, 331, 333. The control console 211 includes a processor 382, coupled to any of the sensors, and configured to process any of the data obtained. Any of these data in its raw form or in a processed form can be displayed on the monitor 350. The control console 211 includes a display 384 and a user interface 386 configured to operate the control console 211. The control console 211 also includes a memory 388 for storing some or all of the obtained data, and some or all of the output of the processor 382. The processor 382 is configured to aggregate the obtained information with one or more groups of saved information. The processor 382 us configured to make comparisons between the obtained information with one or more groups of saved information. In some embodiments, the processor 382 comprises a clock 383 configured to enable the processor 382 to make calculations that utilize time information, both real time and differential time.

Any movement of the patient 380 can be measured and recorded/stored in memory 388. Any angulation of the patient 380 can be measured from the patient 380 themself, or from the angulation of components of the platform, chair, table, or bed 381 on which the patient 380 is placed or carried.

A storage cabinet 212 having one or more storage bins, chambers, or drawers 213, 214, 215, 216 is illustrated in FIG. 1 within the side room 316 associated with the operating room 300. The storage cabinet 212 can in some cases be the main location of supplies used in a medical procedure, including standard supplies (gauze, suture, clamps, anesthesia or medicine bottles) or specialty supplies (implants, instruments, or other medical devices). The movement of a medical personnel, such as personnel 310, between the main room 314 and the side room 316, and more particularly next to the storage cabinet 212, back and forth, can in some procedures occur quite often. Where to best locate the storage cabinet 212 for efficiency purposes may vary, depending on the type of procedure being performed. The optimized location of the storage cabinet 212 can be determined from the processed data taken from the received data from one or more of the sensors or sensor components 318, 320, 322, 324, 334, 336, 338, 340, 342, 344. The storage cabinet 212 can then be moved, accordingly, to optimize any subsequent procedures, or at least any subsequent procedures that are similar for any reason to the procedure just completed, or any subset of previous procedures of a particular type or having one or more common characteristic.

The microphones 334, 336 can also be configured to record speech 390, 392, 394 from one or more of the medical personnel 302, 304, 306, 308, 310. Entire conversations during a medical procedure can be recorded by the microphones 334, 336 and delivered to the console 211, and then processed by the processor 382. For example, key words, phrases, commands, questions, or comments, can be compared by the processor 382 to stored verbal data in the memory 388. Certain words or strings of words (verbal data) may be useful in identifying and modifying the manner in which a medical procedure is performed, or the manner in which the operating room 300 is organized. For example, “hurry up” or “can you please hurry?” can be utilized as a marker for a key bottleneck in the procedural process. “We need more gauze” can help indicate that a larger supply of gauze should have been kept near the patient 380 at the start of the procedure. “I need you here” can indicate that the “floater” (person who moves to two or more areas throughout the procedure) should be replaced by two people. The total person-meters of the procedure are measured, and are the total meters of movement of each personnel. The system 312 is configured to eavesdrop on all of the conversations during the procedure, in the operating room 300 or even outside of it. The obtained verbal data can also be sent to an Artificial Intelligence (AI) unit 396 that is configured to adjust and manipulate the procedure, including commands, equipment setup, personnel setup, personal utilization, personnel training, and length of procedural steps. In some embodiments, the AI unit 396 can be configured to ignore or erase any obviously personal discussions (e.g., any discussions not related to the procedure or to work in general). However, certain seemingly personal phrases can be tagged as being relevant, such as “what time are you going to get sandwiches?” or “I'm getting tired,” as they may have a bearing on scheduling or other practical matters.

The system 312 is configured to use the Artificial Intelligence (AI) unit 396 to make particular discoveries that are not obvious or even noticed by the personnel performing the procedures day in and day out. For example, the AI unit 396 can recognize that to procedures move slower and take longer on Wednesdays than during the rest of the week. Furthermore, the AI unit 396 may discover that the “Wednesday effect” is due to slight changes in the setup that can be traced to maintenance that occurs on Tuesday nights. The system 312 then produces an output in the form of a change report or the equivalent that describes a manner to improve the situation (additional training to the maintenance team or an additional setup step only Wednesday mornings). The change report can be written (e.g., paper) or can be coded (e.g., digital data). Depending on the type of output, the output can be configured to automatically change the setup or the procedure. For example, the output can automatically adjust the settings on one or more pieces of equipment. Overall, the AI unit 396 can serve to “dial in” procedural outcome, for example, to better match targets that the hospital or medical facility might have developed.

In addition to the measurement of verbal data from the speech of the personnel 302, 304, 306, 308, 310, the microphones 334, 336 can also be configured to record non-verbal audio data from the operating room 300 and its environs. For example, error or safety beeps or buzzers from medical equipment can be recorded. The movement of personnel that is measured by the system 312 can even help determine when certain personnel should be relieved by other personnel. For example, the scheduled shift times and durations can be stored on the memory 388.

In some embodiments, multiple sensor types (e.g., camera AND microphone) can be utilized to produce composite data. The composite data can use data relationships in order to provide deeper and more detailed information. For example, a particular movement identified within a video file can be tied or locked to a particular sound within a sound file. For example, a video image of a shoe contacting the floor, may correspond with the click sound.

The recording and processing of information can also provide a real-time status of important safety factors. For example, by simply eavesdropping during the procedure with cameras 318, 320, 322, 324, microphones 334, 336, and/or other sensors, it can be determined that the number of cotton swabs (gauze) that go in to the patient have also been taken out of the patient. In some cases, the medical personnel 302, 304, 306, 308, 310 can even actively support this by stating, out loud, “one gauze in,” “two gauze out,” etc. The same sort of control can be performed, either with eavesdropping by the system 312 alone, or with verbal aid from the medical personnel 302, 304, 306, 308, 310, for control of the number of saline bags used or the number of blood units used. For example, the processor 382, can utilize one or more camera 318, 320, 322, 324 to keep track of the number of blood units that have been used in the procedure, by being active on a count each time a blood bag is visually identified (by comparing to visual data in the memory 388) and then recording a count (e.g., “one”) when the bag is visually identified as being emptied and/or removed from an IV pole. The processor 382 can even make a comparison to current inventory, stored on the memory 388, and then determine when it is time to reorder and blood bag or other item, sending an order list to the inventory control personnel after the procedure. In some cases, non-verbal noises or alternative movements can be utilized by the personnel 302, 304, 306, 308, 310, as described further below.

The system 312 can be configured to produce reports that do not have any personal information about any of the personnel 302, 304, 306, 308, 310, for example, that do not have the name of the person, or any ID numbers, or home address or phone numbers. In some embodiments, the system 312 is configured to obtain all of the information without any of the personnel 302, 304, 306, 308, 310 being aware or able to know that the information is being obtained. Thus, the eavesdropping occurs in a secretive manner. The data obtained can be parsed or otherwise processed by the processor 382, and certain portions placed into the memory 388 or a separate memory such that the data can be supplied to a data mining source. With the combination of the removal of all personal information and the combined data for the data mining source, a safe, secure, means for potentially profiting from what is learned during the procedure is made possible. The information may even indicate certain types of products that don't currently exist, but if they existed would be useful.

In some embodiments, a computer readable storage medium that is not a transitory signal comprises instructions executable by at least one processor to survey a first signal related to information wirelessly obtained by one or more sensor from a medical procedure area, to identify one or more descriptors from the survey of the first signal, and to output a report describing one or more changes related to operation of the medical procedure area based at least in part on the one or more descriptors.

In alternative or adjunctive arrangements, the system 312 can include one or more hand-held remote devices 398, such as a smartphone or pad, that allow the personnel 308 to control the system 312 instead of using the user interface 386 and view information on the remote device 398 instead of on the display 384. In some embodiments, one or more of the personnel 302, 304, 306, 308, 310 can wear facial covers or facial shields 303, 305, 307, 309, 311, including those used in personal protection systems (PPE), wither sterile or non-sterile. The facial covers or shields 303, 305, 307, 309, 311 can provide one-way or two-way communication, and can be configured to engage with the system 312, either as a display (e.g., projected inside the shield with a projector) or as a user interface (e.g., an internal microphone and/or loudspeaker). The display 399 of the remote device 398 can allow the personnel 308 to see information controlled by an application (app) carried on the remote device 398 that is designed for interfacing with the system 312, with a simplified user interface, or even with the equivalent to the entire user interface 386.

While the foregoing is directed to embodiments of the present disclosure, other and further embodiments may be devised without departing from the basic scope thereof. Other embodiments of protection devices having hoods, shrouds bonnets or cuffs, including those having one or two-way communication capability, may be incorporated into the embodiments described herein, such as those described in co-owned International Application Pub. No. WO2021/183984 to PABBAN DEVELOPMENT, INC. et al., published Sep. 16, 2021, which is hereby incorporated by reference in its entirety for all purposes. In some embodiments, commands (advertent or inadvertent) from the user, which are recognized by the system 312, can comprise any one or more of the following: head movement, eyelash movement, eyelid movement, eye movement, nose movement, facial skin movement, mandible movement, ear movement, tongue movement, lip movement, breath flow, mouth heat, breath heat. Command sounds of non-verbal mouth noise advertent or inadvertent) can include one or more of the following: clicks, pops, puffs, hisses, sniffs, coughs, sibilance, whistles, and gurgles.

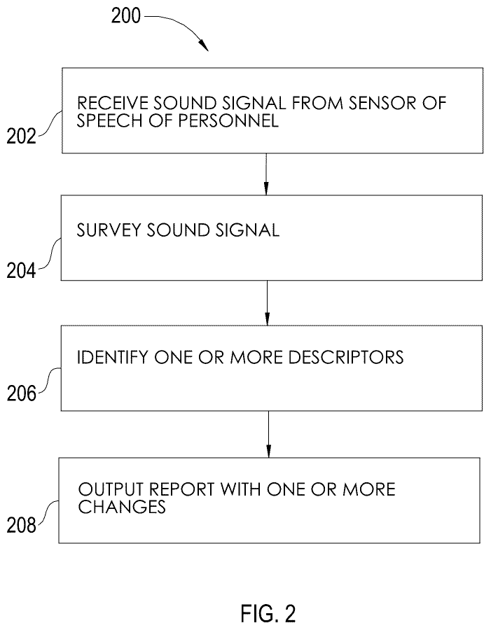

FIG. 2 illustrates a process 200, including step 202, in which a processor 382 receives a signal from one or more microphone 334, 336, wherein the signal represents sound from speech of one or more medical personnel 302, 304, 306, 308, 310. In step 204, the processor 382 surveys the signal. In step 206, the processor 382 identifies one or more descriptors from the surveyed signal. The one or more descriptors can comprise certain sound patterns, or certain text strings, or certain tagged words or tagged phrases, or one or more phenome. In some embodiments, the descriptors can be compared by the processor 382 to stored verbal data in the memory 388. In step 208, the processor outputs a report describing one or more changes. The changes can relate to a medical procedure and/or a medical procedure area.

The following clauses include examples of apparatus of the disclosure.

Clause 1: In one example, a system for improving efficiency in a medical facility includes, one or more sensors configured to wirelessly obtain information from a medical procedure area and output a first signal related to the obtained information, a processor configured to survey the first signal and identify one or more descriptors in the signal, and an output configured to describe one or more changes related to operation of the medical procedure area based at least in part on the one or more descriptors.

Clause 2: In some examples, the output of the system of clause 1 includes a digital report.

Clause 3: In some examples, the output of the system of clause 1 includes a paper report.

Clause 4: In some examples, the output of the system of any one of clauses 1-3 includes a written report.

Clause 5: In some examples, the output of the system of any one of clauses 1-4 includes a coded report.

Clause 6: In some examples, the output of the system of any one of clauses 1-5 automatically causes the one or more changes to occur.

Clause 7: In some examples, the system of any one of clauses 1-6 includes a microphone.

Clause 8: In some examples, the one or more sensors of the system of any one of clauses 1-7 includes a camera.

Clause 9: In some examples, the one or more sensors of the system of any one of clauses 1-8 includes an RFID reader.

Clause 10: In some examples, the one or more sensors of the system of any one of clauses 1-9 includes a receiver.

Clause 11: In some examples, the one or more sensors of the system of any one of clauses 1-10 includes a transceiver.

Clause 12: In some examples, the one or more sensors of the system of any one of clauses 1-11 includes a GPS tracker.

Clause 13: In some examples, the one or more sensors of the system of clause 12 is configured to identify the location of a person.

Clause 14: In some examples, the processor of the system of any one of clauses 1-13 is configured to aggregate the obtained information with one or more groups of saved information.

Clause 15: In some examples, the processor of the system of clause 14 is configured to make comparisons between the obtained information and at least one of the one or more groups of saved information.

Clause 16: In some examples, the processor of the system of clause 15 includes a clock, and wherein the comparisons utilize the clock.

Clause 17: In some examples, the system of any one of clauses 1-16 further includes a remote device containing an application configured to communicate with the processor.

Clause 18: In some examples, the obtained information of the system of either one of clauses 15 or 16 includes one or more voice conversations.

Clause 19: In some examples, the one or more voice conversations of the system of clause 18 are between one or more persons performing a medical procedure in the medical procedure area.

Clause 20: In some examples, the obtained information of the system of clauses 15 or 16 describes movement of one or more persons providing aid to the performance of a medical procedure in the medical procedure area.

Clause 21: In some examples, the obtained information of the system of clause 20 describes movement of the one or more persons out of the medical procedure area.

Clause 22: In some examples, the obtained information of the system of clause 20 describes movement of the one or more persons into the medical procedure area.

Clause 23: In some examples, the obtained information of the system of clause 20 describes movement of the one or more persons from a first location to a second location.

Clause 24: In some examples, the first location of clause 23 is in the vicinity of a procedural table or bed.

Clause 25: In some examples, the second location of either one of clauses 23 or 24 location is in the vicinity of a material storage area.

Clause 26: In some examples, the obtained information of the system of any one of clauses 20-25 describes movement of two or more persons providing aid to the performance of a medical procedure in the medical procedure area.

Clause 27: In some examples, the obtained information of the system of any one of clauses 15-16 or 18-26 describes the presence or lack of presence of one or more surgical instruments in the medical procedure area.

Clause 28: In some examples, the obtained information of the system of any one of clauses 15-16 or 18-26 describes the presence or lack of presence of one or more surgical implants in the medical procedure area.

Clause 29: In some examples, the processor of the system of any one of clauses 1-28 is configured to relate verbal information with non-verbal information.

Clause 30: In some examples, the verbal information of clause 29 includes a particular comment by a first person and wherein the non-verbal information includes at least one characteristic selected from the list consisting of: order of operation steps, length in time of one or more operation steps, location of one or more surgical instruments, location of one or more surgical implants, unpackaging of one or more surgical instruments, unpackaging of one or more surgical implants, location of a second person, location of the first person, movement of the second person, movement of the first person, and location of a storage area.

Clause 31: In some examples, the processor of the system of any one of clauses 1-30 includes an artificial intelligence (AI) apparatus.

Clause 32: In some examples, the processor of the system of any one of clauses 1-18 or 20-26 is configured to cross-reference a database including stored data related to a medical procedure.

Clause 33: In some examples, the processor of the system of clause 32 includes an artificial intelligence (AI) apparatus.

Clause 34: In some examples, the processor of the system of either one of clauses 32 or 33 is configured to compare the stored data with a characteristic from the list consisting of: time, efficiency, and procedural outcome.

Clause 35: In another example, computer readable storage medium that is not a transitory signal includes instructions executable by at least one processor to: survey a first signal related to information wirelessly obtained by one or more sensor from a medical procedure area, identify one or more descriptors from the survey of the first signal, and output a report describing one or more changes related to operation of the medical procedure area based at least in part on the one or more descriptors.

Clause 36: In some examples, the outputted report of clause 35 includes a digital report.

Clause 37: In some examples, the outputted report of clause 35 includes a paper report.

Clause 38: In some examples, the outputted report of any one of clauses 35-37 includes a written report.

Clause 39: In some examples, the outputted report of any one of clauses 35-38 includes a coded report.

Clause 40: In some examples, the outputted report of any one of clauses 35-39 automatically causes the one or more changes to occur.

Clause 41: In some examples, the one or more sensors of any one of clauses 35-40 includes a microphone.

Clause 42: In some examples, the one or more sensors of any one of clauses 35-41 includes a camera.

Clause 43: In some examples, the one or more sensors of any one of clauses 35-42 includes an RFID reader.

Clause 44: In some examples, the one or more sensors of any one of clauses 35-43 includes a receiver.

Clause 45: In some examples, the one or more sensors of any one of clauses 35-44 includes a transceiver.

Clause 46: In some examples, the one or more sensors of any one of clauses 35-45 includes a GPS tracker.

Clause 47: In some examples, the one or more sensors of clause 46 is configured to identify the location of a person.

Clause 48: In some examples, the processor of any one of clauses 45-47 is configured to aggregate the obtained information with one or more groups of saved information.

Clause 49: In some examples, the processor of clause 48 is configured to make comparisons between the obtained information and at least one of the one or more groups of saved information.

Clause 50: In some examples, the processor of clause 49 includes a clock, and wherein the comparisons utilize the clock.

Clause 51: In some examples, any one of clauses 45-50 further includes a remote device containing an application and configured to communicate with the processor.

Clause 52: In some examples, the obtained information of either one of clauses 49 or 50 includes one or more voice conversations.

Clause 53: In some examples, the one or more voice conversations of clause 52 are between one or more persons performing a medical procedure in the medical procedure area.

Clause 54: In some examples, the obtained information of either one of clauses 49 or 50 describes movement of one or more persons providing aid to the performance of a medical procedure in the medical procedure area.

Clause 55: In some examples, the obtained information of clause 54 describes movement of the one or more persons out of the medical procedure area.

Clause 56: In some examples, the obtained information of clause 54 describes movement of the one or more persons into the medical procedure area.

Clause 57: In some examples, the obtained information of clause 54 describes movement of the one or more persons from a first location to a second location.

Clause 58: In some examples, the storage medium of clause 57, includes wherein the first location is in the vicinity of a procedural table or bed.

Clause 59: In some examples, the storage medium of either one of clauses 57 or 58 includes wherein the second location is in the vicinity of a material storage area.

Clause 60: In some examples, the storage medium of any one of clauses 54-59 includes wherein the obtained information describes movement of two or more persons providing aid to the performance of a medical procedure in the medical procedure area.

Clause 61: In some examples, the storage medium of any one of clauses 49-50 or 52-60 includes wherein the obtained information describes the presence or lack of presence of one or more surgical instruments in the medical procedure area.

Clause 62: In some examples, the storage medium of any one of clauses 49-50 or 52-60 includes wherein the obtained information describes the presence or lack of presence of one or more surgical implants in the medical procedure area.

Clause 63: In some examples, the storage medium of any one of clauses 35-62 includes wherein the processor is configured to relate verbal information with non-verbal information.

Clause 64: In some examples the storage medium of clause 63 includes wherein the verbal information includes a particular comment by a first person and wherein the non-verbal information includes at least one characteristic selected from the list consisting of: order of operation steps, length in time of one or more operation steps, location of one or more surgical instruments, location of one or more surgical implants, unpackaging of one or more surgical instruments, unpackaging of one or more surgical implants, location of a second person, location of the first person, movement of the second person, movement of the first person, and location of a storage area.

Clause 65: In some examples the storage medium of any one of clauses 35-64 includes wherein the processor includes an artificial intelligence (AI) apparatus.

Clause 66: In some examples, the storage medium of any one of clauses 35-52 or 54-60 include wherein the processor is configured to cross-reference a database including stored data related to a medical procedure.

Clause 67: In some examples, the storage medium of clause 66 includes wherein the processor includes an artificial intelligence (AI) apparatus.

Clause 68: In some examples, the storage medium of either one of clauses 66 or 67 includes wherein the processor is configured to compare the stored data with a characteristic from the list consisting of: time, efficiency, and procedural outcome.

Clause 69: In still another example, a method for improving efficiency in a medical facility includes surveying a first signal related to information wirelessly obtained by one or more sensor from a medical procedure area, identifying one or more descriptors from the survey of the first signal, and outputting a report describing one or more changes related to operation of the medical procedure area based at least in part on the one or more descriptors.

Clause 70: In some examples, the method of clause 69 includes wherein the outputted report includes a digital report.

Clause 71: In some examples, the method of clause 69 includes wherein the outputted report includes a paper report.

Clause 72: In some examples, the method of any one of clauses 69-71 includes wherein the outputted report includes a written report.

Clause 73: In some examples, the method of any one of clauses 69-72 includes wherein the outputted report includes a coded report.

Clause 74: In some examples, the method of any one of clauses 69-73 includes wherein the outputted report automatically causes the one or more changes to occur.

Clause 75: In some examples, the method of any one of clauses 69-74 includes wherein the one or more sensors includes a microphone.

Clause 76: In some examples, the method of any one of clauses 69-75 includes wherein the one or more sensors includes a camera.

Clause 77: In some examples, the method of any one of clauses 69-76 includes wherein the one or more sensors includes an RFID reader.

Clause 78: In some examples, the method of any one of clauses 69-77 includes wherein the one or more sensors includes a receiver.

Clause 79: In some examples, the method of any one of clauses 69-78 includes wherein the one or more sensors includes a transceiver.

Clause 80: In some examples, the method of any one of clauses 69-79 includes wherein the one or more sensors includes a GPS tracker.

Clause 81: In some examples, the method clause 80 includes wherein the one or more sensors is configured to identify the location of a person.

Clause 82: In some examples, the method of clause 81 further includes aggregating the obtained information with one or more groups of saved information via a processor.

Clause 83: In some examples, the method of clause 82 further includes making comparisons between the obtained information and at least one of the one or more groups of saved information via the processor.

Clause 84: In some examples, the step of making comparisons of clause 83 further includes utilizing a clock contained on the processor.

Clause 85: In some examples, the method of any one of clauses 82-84 further includes utilizing a remote device containing an application to communicate with the processor.

Clause 86: In some examples, the method of any one of clauses 83 or 84 further includes wherein the obtained information includes one or more voice conversations.

Clause 87: In some examples, the method of clause 86 further includes wherein the one or more voice conversations are between one or more persons performing a medical procedure in the medical procedure area.

Clause 88: In some examples, the method of any one of clauses 83 or 84 further includes wherein the obtained information describes movement of one or more persons providing aid to the performance of a medical procedure in the medical procedure area.

Clause 89: In some examples, the method of clause 88 includes wherein the obtained information describes movement of the one or more persons out of the medical procedure area.

Clause 90: In some examples, the method of clause 88 includes wherein the obtained information describes movement of the one or more persons into the medical procedure area.

Clause 91: In some examples, the method of clause 88 includes wherein the obtained information describes movement of the one or more persons from a first location to a second location.

Clause 92: In some examples, the method of clause 91 includes wherein the first location is in the vicinity of a procedural table or bed.

Clause 93: In some examples, the method of any one of clauses 91 or 92 further includes wherein the second location is in the vicinity of a material storage area.

Clause 94: In some examples, the method of any one of clauses 88-93 includes wherein the obtained information describes movement of two or more persons providing aid to the performance of a medical procedure in the medical procedure area.

Clause 95: In some examples, the method of any one of clauses 83-84 or 86-94 further includes wherein the obtained information describes the presence or lack of presence of one or more surgical instruments in the medical procedure area.

Clause 96: In some examples, the method of any one of clauses 83-84 or 86-94 further includes wherein the obtained information describes the presence or lack of presence of one or more surgical implants in the medical procedure area.

Clause 97: In some examples, the method of any one of clauses 82-96 further includes relating verbal information with non-verbal information via the processor.

Clause 98: In some examples, the method of clause 97 further includes wherein the verbal information includes a particular comment by a first person and wherein the non-verbal information includes at least one characteristic selected from the list consisting of: order of operation steps, length in time of one or more operation steps, location of one or more surgical instruments, location of one or more surgical implants, unpackaging of one or more surgical instruments, unpackaging of one or more surgical implants, location of a second person, location of the first person, movement of the second person, movement of the first person, and location of a storage area.

Clause 99: In some examples, the method of any one of clauses 82-96 further includes wherein the surveying includes eavesdropping.

Clause 100: In some examples, the method of any one of clauses 69-99 further includes wherein the surveying and the identifying are not perceptible to any person within the medical procedure area.

Clause 101: In some examples, the method of any one of clauses 69-100 further includes wherein the outputting creates a report with no person's personal identification.

Clause 102: In some examples, the method of any one of clauses 69-101 further includes wherein no person's personal identification includes no person's name.

Clause 103: In some examples, the method of any one of clauses 69-102 further includes wherein no person's personal identification includes no patient's name.

Clause 104: In some examples, the method of any one of clauses 69-102 further includes wherein no person's personal identification includes no medical personnel's name.

Clause 105: In some examples, the method of any one of clauses 82-98 further includes wherein the processor includes an artificial intelligence (AI) apparatus.

Clause 106: In some examples, the method of any one of clauses 82-86 further includes wherein the processor is configured to cross-reference a database including stored data related to a medical procedure.

Clause 107: In some examples, the method of clause 106 further includes wherein the processor includes an artificial intelligence (AI) apparatus.

Clause 108: In some examples, the method of either one of clauses 106 or 107 further includes using the processor to compare the stored data with a characteristic from the list consisting of: time, efficiency, and procedural outcome.

The disclosure further relates to heads-up displays for use with facial covers or facial shields, including those used in personal protection systems, including, but not limited to personal environmental protections systems. The heads-up displays can be used to aid in the optimization of medical procedures, including surgeries. The personal protections systems often include a headgear structure which is worn by an individual to protect from particulate material. The personal protection systems can provide filtered air to the user. The disclosure also relates to devices, apparatus or methods for life-saving, including devices for medical use. The disclosure also relates to respirators or can relate to respiratory apparatus, such as respiratory apparatus for medical purposes, including apparatus with filter elements. The disclosure also describes information systems that couple information related to the performance and optimization of a medical procedure with a facial shield-provided heads-up display of a user.

There are several types of air flow, filtration and protective systems which are known in the art. Several types of such systems are currently available on the market for use in surgical arenas, in “clean room” environments, or in hazardous/contaminated environments.

Some of the existing systems include hoods, gowns, filters, and the like. In some instances, the air filters are built into the helmet structure. Known units frequently include external sources of air such as gas cylinders, air lines or the like which are connected to the helmet structure by tubes, hoses or the like.

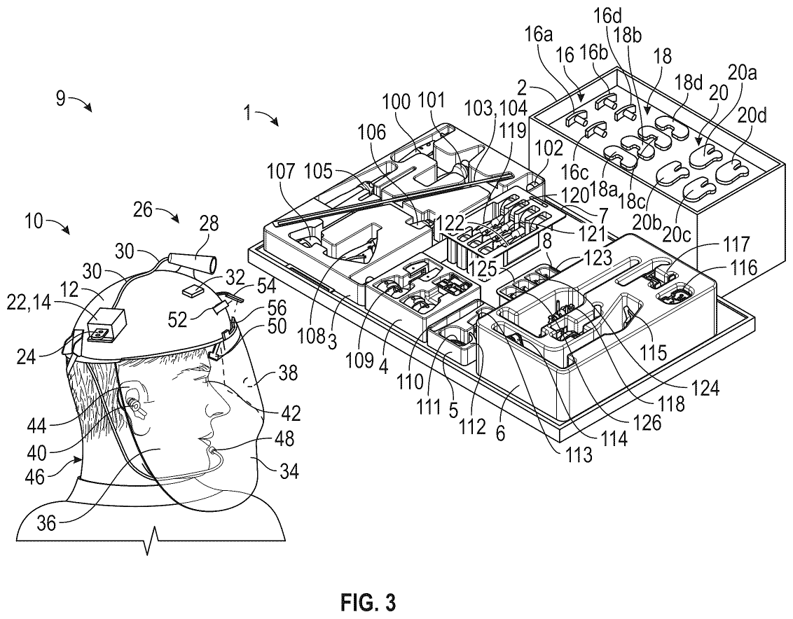

In FIG. 3, a surgeon 10 in an operating room 9 wears a helmet 12 or other type of head support. The helmet 12 is configured to carry a control system 14 which is configured to interface with a surgical kit 1. The surgical kit 1 includes an instrumentation container 2 and several support containers 3, 4, 5, 6, 7, 8. The surgical kit 1 can in alternative embodiments comprise a kit configured for any type of surgery or other medical procedure, but the surgical kit 1 of FIG. 1 comprises a kit for a total knee replacement surgery. The instrumentation container 2, includes one or more tibial components 16, one or more tibial bearings 18, and one or more femoral components 20.

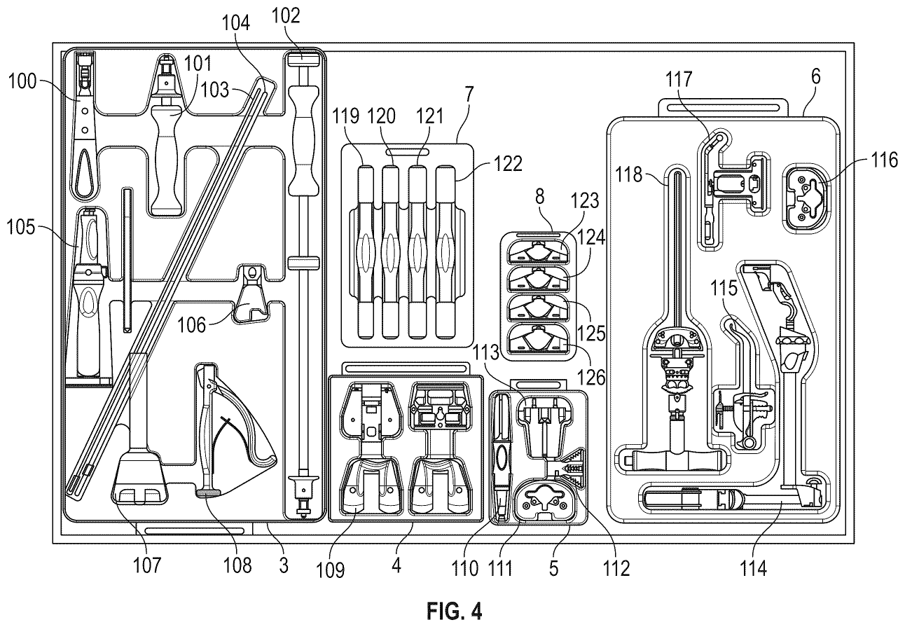

As shown in FIG. 3 and FIG. 4, an instrument container 3 includes instruments such as a tibial component handle component handle 100, a universal handle 101, a slap hammer 102, alignment rods 103, 104, a tibial punch handle 105, a tibial impactor 106, a femoral notch impactor 107, and a pin puller 108. An instrument container 4 includes additional instruments 109. An instrument container 5 includes instruments such as a tibial stem drill 110, a tibial template 111, a tibial punch 112, and a tibial drill punch guide 113. A primary cuts container 6 includes a tibial resection jig 114, a tibial stylus 115, a tibial sizing device 116, a femoral sizing and rotation device 117, and a distal femoral resection jig 118. A spacer block container 7 includes spacer blocks 119, 120, 121, 122 of different sizes, for example 10 mm, 12.5 mm, 15 mm, and 17.5 mm. A tibial trial container 8 includes tibial trial inserts 123, 124, 125, 126 of different thicknesses, for example 10 mm, 12.5 mm, 15 mm, and 17.5 mm.

The control system 14 is configured to aid the surgery by allowing the surgeon 10 to immediately identify each of the instruments 100-126 and each of the instrumentation components 16a-d, 18a-d, 20a-d. The control system 14 includes one or more components carried on the helmet 12, carried by the surgeon 10, or located in the vicinity of the surgeon 10 and/or the operating room 9, such that information from the instruments 100-126 and/or the instrumentation components 16a-d, 18a-d, 20a-d can be wirelessly obtained. The control system 14 includes a control box 22 containing a controller 24, and coupled to one or more sensors 26, which can also be considered a part of the control system 14. The one or more sensors 26 include a camera 28, which is connected to the control box 22 by a cable 30, which can include electrical wires and/or fiberoptics. The one or more sensors 26 can also include a receiver 32. The one or more sensors 26 are carried on the helmet 12, and are configured to, independently or in combination, wirelessly receive information from the instruments 100-126 and/or from the instrumentation components 16a-d, 18a-d, 20a-d. In some embodiments, the receiver 32 comprises a transceiver and is configured to send information to any one or more of the instruments 100-126 and/or to any one or more of the instrumentation components 16a-d, 18a-d, 20a-d.

Before, during, and/or after a surgery, a surgeon 10 (or any other medical personnel) desires to be fully correct and complete in the performance of every element of the procedure. Thus, it is important to know as much as possible about each instrument 100-126 and each instrumentation component 16a-d, 18a-d, 20a-d. Oftentimes, a sales representative and/or a clinical specialist from the company whose kit 1 is being used is present during the procedure, and is available to answer questions regarding the specifics of each instrument 100-126 or instrumentation component 16a-d, 18a-d, 20a-d. It is common that this company employee is relied upon by surgical staff in order to complete the complex procedure within minimal confusion or delay. However, this is not an ideal situation. Hospitals or surgical centers do not typically employ these industry representatives, either directly or indirectly. The representatives are present for the purpose of making sure their products are used correctly, but their main purpose is to make sure that their products are being used, thus maintaining current and future sales. It does sometimes occur that a technologist, assistant, nurse, or other physician learns a large number of pertinent details about the instruments 100-126 and instrumentation components 16a-d, 18a-d, 20a-d, and is thus able to answer the required questions. However, the industry representative is quite often the person present at the procedure that is the most up-to-date on the details regarding instruments 100-126 and instrumentation components 16a-d, 18a-d, 20a-d, and sometimes even the procedure itself, at least from a logistical perspective.

However, it is not always the case that the industry representative or the in-house expert is available for every portion of every procedure. Some hospitals have instigated rules to limit the frequency that an industry representative can be present. The Covid-19 pandemic has also caused hospitals to initiate policies to limit or further limit the number of people present in a surgical procedure. Thus, the reliance on particular people, within or without, to provide key information to the team cannot be consistently assured. Furthermore, with so many components being utilized, even an expert may not have all of the information necessary, and may sometimes even deliver incorrect information. The information can include implant size, material, bar code, function, compatibility or fit of one implant with another implant, which instrument to use with which implant model, and how to perform the procedure with each implant or instrument. The procedure can indeed vary, depending on which implant is chosen, or which instrument is used. In additional, certain clinical conditions, either patient-related, environment-related, or procedure-related, can indicate which implant to use or which instrument to use, or which manner to implant each implant, or which manner to use each instrument.

The control system 14 allows the surgeon 10 to effortlessly perform the surgical procedure, and learn important information about and select the desired instruments 100-126 and/or the instrumentation components 16a-d, 18a-d, 20a-d, without requiring aid from any other person, and without having to touch anything with any part of their body or garments/gloves. The control system 14 is also configured to automatically input the selected instruments 100-126 and/or the instrumentation components 16a-d, 18a-d, 20a-d into the hospital records being recorded about the surgical procedure. By relying on a continually updated database, the information obtained is of the most reliable quality.

A facial shield 34 comprises a substantially clear polymeric sheet and is configured to be detachably coupled to the helmet 12. The helmet 12 and facial shield 34 can in some embodiments comprise a PAPR system (Powered Air Purifying Respirator), to substantially control the surgeon's 10 breathing environment via air filtration, inflow, and/or outflow, and can include some or all features of any of the embodiments described in U.S. Pat. No. 8,302,599 to Green issued Nov. 6, 2012, and entitled “Protective Headgear System with Filter Protector,” which is incorporated herein by reference in its entirety for all purposes. However, in other embodiments, the helmet 12 and facial shield 34 serve the general purpose of protecting the face 36 of the surgeon 10 for direct exposure of particulate, splash, or any other type of contamination, without the air control provided by PAPR. The facial shield 34 comprises a display screen portion 38 configured to allow the surgeon 10 to visualize with one or both eyes 42 information received related to the instruments 100-126 and/or the instrumentation components 16a-d, 18a-d, 20a-d in either text or figure form. One or more earbuds 40 within the surgeon's ear(s) 44 provide an additional or alternative audible form of the text relating to the information received.

The display screen portion 38 of the facial shield 34 is configured to provide a heads-up display (HUD) for the surgeon 10. The camera 28 is positioned such that it receives an image of any object that is in the center of vision of the wearer of the helmet 12 (the surgeon 10). Thus, when the surgeon 10 views one of the instruments 100-126 or one of the instrumentation components 16a-d, 18a-d, 20a-d and orients the head 46/helmet 12 such that the image of the instrument 100-126 or component 16a-d, 18a-d, 20a-d of interest is centered within the display screen portion 38, the processor 24 compares the image to an image database using recognition software. The identified product (model number, etc.) contains pertinent data relating thereto, and some or all of this data is displayed on the display screen portion 38 by a projector 52. The projector 52 can project onto the display screen portion 38 directly, or (as shown) via a first mirror 54, a second mirror 56, or additional mirrors. In some embodiments, the projector 52 comprises a light emitting diode (LED) projector. In some embodiments, the projector 52 comprises a liquid crystal display (LCD) projector. In some embodiments, the projector 52 comprises a short throw or ultra-short throw projector. In some embodiments, the surgeon 10 can advance displayed images (e.g., one, two, three, etc.) by voice commends spoken into a microphone 48 that is coupled to the processor 24. The processor 24 can be configured to recognize abbreviated terms, and to sense language even when whispered. The processor 24 can be trained to only recognize commands from a particular user's voice. In some embodiments, the processor 24 can be programmed to understand standard spoken language in a variety of languages; for example, “tell me the different sizes available for the currently selected device.” Alternatively, the surgeon can give commands using quiet or silent (non-verbal) facial movement only (eyelid blinks, eye movement, lip movement, nose movement) that is sensed by a motion sensor 50 that is coupled to the processor 24. Commands can literally be: SELECT, DESELECT, NEXT. In other embodiments, the commands can be other terms, such as: YES, NO, LAST, NEXT, FIRST. In some embodiments, the camera 28 is configured to calculate a distance between the surgeon 10 and a chosen instrument 100-126 or component 16a-d, 18a-d, 20a-d or between one instrument 100-126 or component 16a-d, 18a-d, 20a-d and another instrument 100-126 or component 16a-d, 18a-d, 20a-d. In some embodiments the camera 28 comprises an infrared camera or can include an additional camera that is an infrared camera.

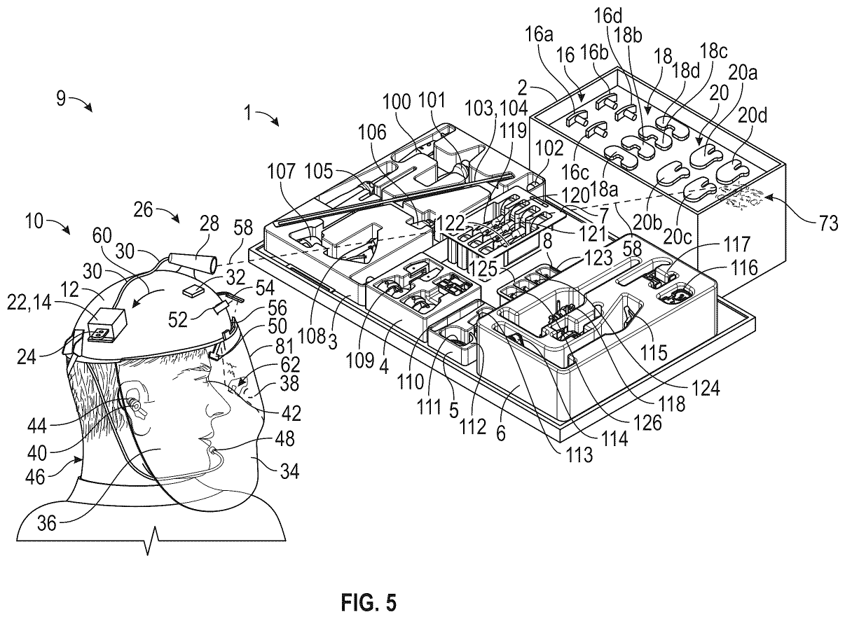

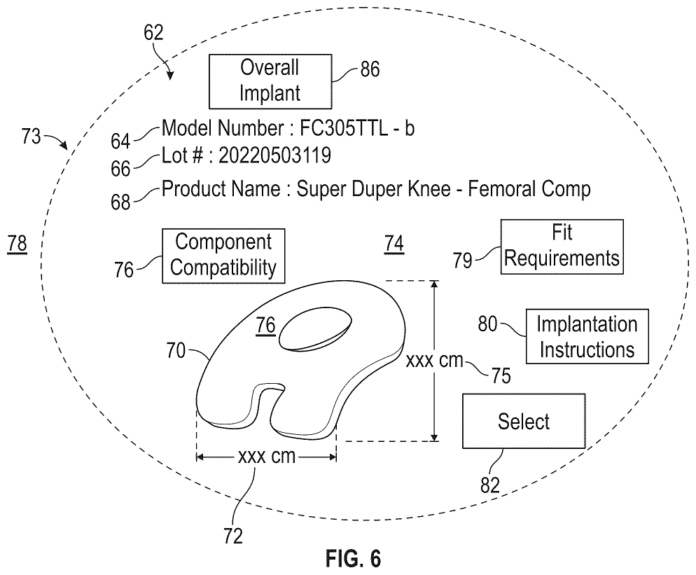

Turning to FIG. 5, the surgeon 10 is shown choosing between femoral components 20a-d. The surgeon 10 has adjusted their head 46, and thereby adjusted the helmet 12 and the camera 28 held thereon and in synchronized movement with, such that the camera 28 has a sight line 58 to femoral component 20c. The processor 24 receives image information 60 (arrow), which is compared to an image database using recognition software. The processor 24 identifies the model number, or other identifier, of the femoral component 20c, and projects an image 62 with the projector 52 onto the display screen portion 38 of the facial shield 34. The image 62, in exemplary form, is shown in FIG. 6. The image 62 includes one or more photo or drawing 70 of the implant (femoral component 20c, in this instance). The photo or drawing 70 includes a first dimension 72 and a second dimension 75 that are specific to this appropriate model of implant. A model number 64, lot number 66, and product name 68 are also listed on the image 62, adjacent the photo or drawing 70. The surgeon 10 can simultaneously also see/look through the clear facial shield 34 and visualize the actual back ground (e.g., the actual instruments 100-126 or components 16a-d, 18a-d, 20a-d and surroundings). The surgeon 10, while doing this, can visualize the background (and components) by looking at and through areas 74 that are free of projection on the display screen portion 38, or at areas 76 that are behind the projected image 62, or at areas 78 that are outside of the display screen portion 38. Thus, the image 62 is overlayed in the surgeon's 10 field of view. In some embodiments, the processor 24 is configured or programmed to overlay the drawing 70 directly over the product (e.g., component 20c). In some embodiments, the processor 24 is configured to flip or turn the image (drawing 70) to match the current orientation of the product in the view captured by the camera 28; and to scale up or down the drawing 70 for the closest size match with the product. In some embodiments, the processor 24 is configured to overlay the drawing 70 adjacent the product (e.g., component 20c), for example side-by-side, or top-to-bottom. In some embodiments, the image 62 and any of the true background can be combined using mixed-reality. In some embodiments, the image 62 can appear three-dimensional or include one or more hologram. In some embodiments, the text 73 can be presented to the user to appear directly over or next to the actual instruments or instrumentation components seen by the user (see ghost image of text 73 in FIG. 5). In some embodiments, augmented reality can be produced in the image. For example, a particular implant can be overlayed onto a patient in the image, and in addition a visual device, such as an instructional overlay, can be added to the image.

The image 62 further includes several activation targets 76, 79, 80 that can be selected by the user (surgeon 10). The activation targets 76, 79, 80 can be configured to be selected by the user by voice activation (microphone 48), or by facial movement (motion sensor 50/facial recognition software). Alternatively, the user (surgeon 10) can touch an external portion 81 of the facial shield 34 behind the activation target of choice 76, 79, 80 (FIG. 5). In some embodiments, the surgeon 10 can utilize a smart watch or other smart device to wirelessly make some or all of the selections. In some embodiments, smart systems such as Siri®, Alexa®, or Google Home™ can be utilized. In some embodiments, the surgeon 10 can receive calls or messages from e-mail, smart phone, or other smart devices, and the controller 24 can provide them visually on the display 62 or aurally through the earbud(s) 40. The “COMPONENT COMPATIBILITY” activation target 76 can activate a second image 62 that is projected onto the display screen portion 38, either together with the first image 62, or replacing the first image 62, the second image 62 listing all of the other components 16a-d, 18a-d, or instruments 100-126 which are compatible with the selected component 20c. Alternatively, an audio version of this list can be played to the surgeon 10 through the one or more earbud(s) 40. In some embodiments, the controller 24 is configured (e.g., programmable) to identify any components or instruments that are missing. For example, components or instruments that would be required or desired for any particular procedure, or any variation of any procedure. In some embodiments, the controller 24 is configured (e.g., programmable) to identify components or instruments that should not be used, but that have (e.g., inadvertently) been placed in the area of interest, such that these components or instruments can be removed from the area of interest and/or removed from any procedure documentation or billing documentation.

The “FIT REQUIREMENTS” activation target 79 can activate a third image 62 that is projected onto the display screen portion 38, either together with the first image 62 and/or second image 62, or replacing the first image 62 and/or second image 62, the third image 62 listing all of the requirements, limitations, or instructions to couple the chosen component 20c to the other components 16a-d, 18a-d, or instruments 100-126. Alternatively, an audio version is played to the surgeon 10 through the one or more earbud(s) 40.

The “IMPLANTATION PROCEDURE” activation target 80 can activate a fourth image 62 that is projected onto the display screen portion 38, either together with the first image 62 and/or second image 62 and/or third image 62, or replacing the first image 62 and/or second image 62 and/or third image 62, the fourth image 62 listing the instructions for implanting the component 20c. Alternatively, an audio version is played to the surgeon 10 through the one or more earbud(s) 40. Though not shown, an alternative activation target (and corresponding image) can correspond to the use of the implants themselves.

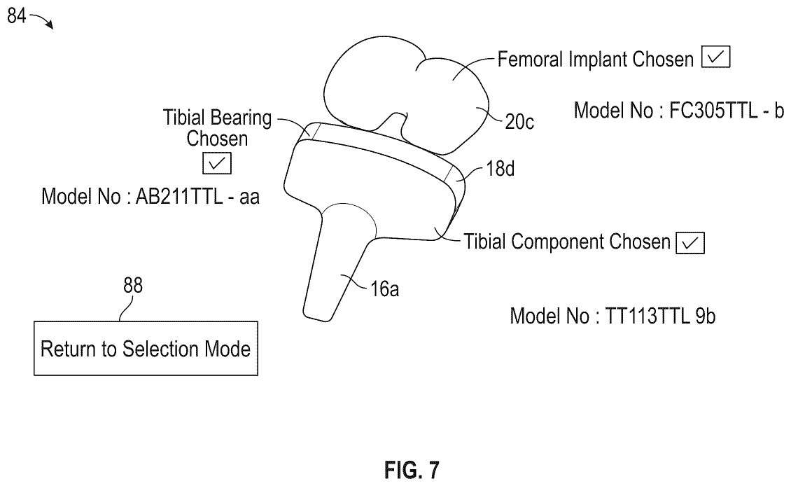

A “SELECT” activation target 82 can be selected by the surgeon 10 when the surgeon 10 makes the decision to use this particular component 20c. The processor 24 saves this information and is configured to produce an overall implant image 84, as shown in FIG. 7. The overall implant image 84 is available via selection from image 62 (FIG. 6) when the surgeon 10 selects the “OVERALL IMPLANT” activation target 86. The surgeon 10 can return to the image 62 of FIG. 6 (e.g., selection mode), by activating the “RETURN TO SELECTION MODE” activation target 88, by any of the manners described. In some embodiments, the surgeon 10 is able to directly select or directly de-select any of the components 16a, 18d, 20c in the overall implant image 84. In some embodiments, the processor 24 is configured or programmed to notify the surgeon 10 if any of the components selected are not compatible with each other. E.g., it can indicate a selection error: “SELECTION ERROR,” for example on the overall implant image 84.

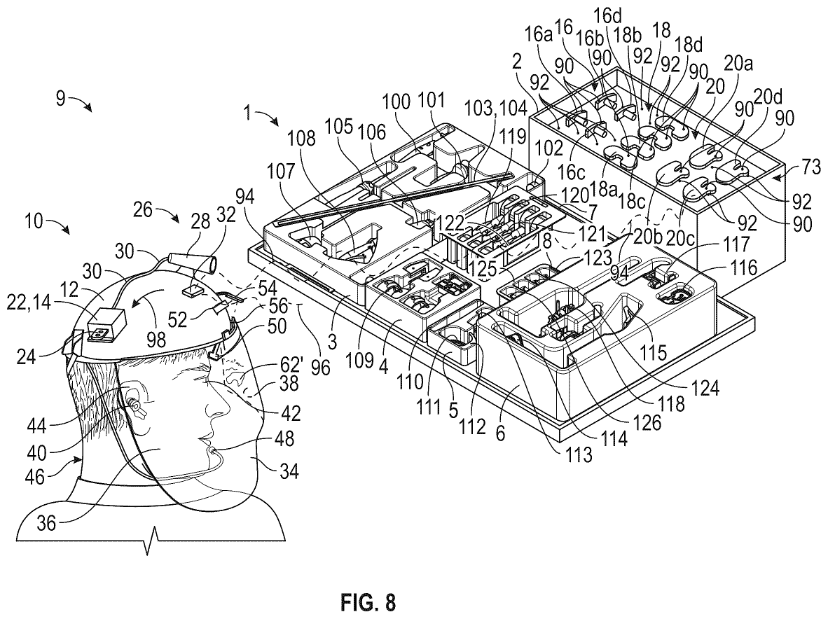

An alternative selection mode is shown in FIG. 8. The surgeon 10 is shown choosing between femoral components 20a-d, by utilizing electromagnetic selection with the receiver 32. The surgeon 10 has adjusted their head 46, and thus adjusted the helmet 12 and receiver 32 held on the head 46 and in synchronized movement with, such that the receiver 32 (e.g., transceiver) is able to receive a wireless communication signal 94 from a transmitter (e.g., transceiver) 90 in the femoral component 20c. Alternatively, the receiver 32 (e.g., transceiver) is able to receive a wireless communication signal 94 from a transmitter (e.g., transceiver) 92 immediately adjacent the femoral component 20c. Other transmitters 90, 92 are shown in the other components 16a-d, 18a-d, 20a,b,d. Also, the instruments 100-126 can include transmitters 90, 92, though they are not shown in FIG. 8. In some embodiments, the transmitters 90, 92 comprise RFID chips that are passive, but are excited/activated from a signal 96 by the transmitter/transceiver 32, and in turn send a signal 94 containing electromagnetic information 98 (arrow). The processor 24 receives information 98, which is compared to an image database using recognition software. The processor 24 identifies the model number, or other identifier, of the femoral component 20c, and projects an image 62′ with the projector 52 onto the display screen portion 38 of the facial shield 34. The image 62′ may be similar to image 62 in FIG. 6. In some embodiments, the processor 24 is configured to identify all of the instruments 100-126 or instrumentation components 16a-d, 18a-d, 20a-d in the vicinity of the surgeon 10. In some embodiments, the processor 24 is configured to automatically (e.g., upon start-up) identify all of the instruments 100-126 or instrumentation components 16a-d, 18a-d, 20a-d in the vicinity of the surgeon 10. In some embodiments, the facial shield 34 is provided sterile. In some embodiments, the facial shield 34 is disposable.

In other embodiments, loudspeakers can replace the transmitters 90, 92, and a microphone can replace the receiver 32, so that sound information is used to transmit the information. In some embodiments, the sound information comprises ultrasound frequencies. In other embodiments, the transmitters 90, 92 and receiver 32 can be replaced by other types of senses and sensors, such as chemical sensors (sniffers).

The system for data gathering and processing 312 can in some embodiments incorporate some or all of the features of the control system 14, and vice versa. In one example, a system for improving efficiency in a medical facility includes, one or more sensors configured to wirelessly obtain information from a medical procedure area and output a first signal related to the obtained information, a processor configured to survey the first signal and identify one or more descriptors in the signal, an output configured to describe one or more changes related to operation of the medical procedure area based at least in part on the one or more descriptors, a cover configured to protect the head of a user from contaminants in an ambient environment external to the cover, the cover comprising a substantially transparent facial shield, a projector configured to project an image on at least a portion of the facial shield that is configured to reside within a field of vision of the user, and an implant information sensor configured to wirelessly obtain information from a medical implant within a medical procedure area and output a second signal related to the obtained information, wherein the processor is further configured to determine an identity characteristic of the implant based on the second signal.

While the foregoing is directed to embodiments of the present disclosure, other and further embodiments may be devised without departing from the basic scope thereof. Other embodiments of protection devices having hoods, shrouds bonnets or cuffs may be incorporated into the embodiments described herein, such as those described in co-owned International Application Pub. No. WO2021/183984 to PABBAN DEVELOPMENT, INC. et al., published Sep. 16, 2021. In some embodiments, commands from the user can comprise any one or more of the following: head movement, eyelash movement, eyelid movement, eye movement, nose movement, facial skin movement, mandible movement, ear movement, tongue movement, lip movement, breath flow, mouth heat, breath heat. Command sounds of non-verbal mouth noise can include one or more of the following: clicks, pops, puffs, hisses, sniffs, coughs, sibilance, whistles, and gurgles.

The following clauses include examples of apparatus of the disclosure.

Clause 109: In one example, system for aiding a medical procedure includes a cover configured to protect the head of a user from contaminants in an ambient environment external to the cover, the cover including a substantially transparent facial shield, a projector configured to project an image on at least a portion of the facial shield that is configured to reside within a field of vision of the user, a sensor configured to wirelessly obtain information from a medical implant within a medical procedure area and output a first signal related to the obtained information, and a processor configured to determine an identity characteristic of the implant based on the first signal.

Clause 110: In some examples, the identity characteristic of clause 109 is a characteristic selected from the list consisting of: an implant type, an implant model, an implant lot, and an implant size.

Clause 111: In some examples, the sensor of either one of clauses 109 or 110 is configured to obtain information selected from the list consisting of: visual information, electromagnetic information, chemical information, and audio information.

Clause 112: In some examples, the sensor of any one of clauses 109-111 includes a camera.

Clause 113: In some examples, the sensor of any one of clauses 109-111 includes a receiver.

Clause 114: In some examples, the sensor of any one of clauses 109-111 includes a microphone.

Clause 115: In some examples, the sensor of any one of clauses 109-111 includes a chemical sensor.

Clause 116: In some examples, the projector of any one of clauses 109-115 includes a light emitting diode display.

Clause 117: In some examples, the projector of any one of clauses 109-115 includes a liquid crystal display.

Clause 118: In some examples, the cover of any one of clauses 109-117 includes a helmet.

Clause 119: In some examples, the cover of any one of clauses 109-117 includes a hood.

Clause 120: In some examples, the cover of any one of clauses 109-117 includes a shroud.

Clause 121: In some examples, the cover of any one of clauses 109-117 includes a bonnet.

Clause 122: In some examples, the cover of any one of clauses 109-117 includes a cuff.

Clause 123: In some examples, the cover of any one of clauses 109-122 is configured to determine the identity characteristic of the implant without requiring the receipt of any verbal command.

Clause 124: In some examples, the cover of any one of clauses 109-122 is configured to determine the identity characteristic of the implant without requiring any verbal command from the user.

Clause 125: In some examples, the cover of any one of clauses 109-124 is configured to receive non-verbal information from the user.

Clause 126: In some examples, the non-verbal information of clause 125 is selected from the list consisting of: head movement, eyelash movement, eyelid movement, eye movement, nose movement, facial skin movement, mandible movement, ear movement, tongue movement, lip movement, breath flow, mouth heat, breath heat.

Clause 127: In some examples, the non-verbal information of clause 125 includes one or more non-verbal mouth noise.

Clause 128: In some examples, the non-verbal mouth noise of clause 128 includes a noise selected from the list consisting of: a click, a pop, a puff, a hiss, a sniff, a cough, a sibilance, a whistle, and a gurgle.

Clause 129: In some examples, the processor of any one of clauses 125-128 is configured to enable the user to make a selection using the non-verbal information.

Clause 130: In some examples, the selection of clause 129 selecting that the implant is the correct implant to use.

Clause 131: In some examples, the sensor of any one of clauses 125-130 is further configured to sense the non-verbal information from the user and to output a second signal related to the non-verbal information.

Clause 132: In some examples, the system of any one of clause 125-130 further includes a non-verbal information sensor configured to sense the non-verbal information from the user and to output a second signal related to the non-verbal information.

Clause 133: In some examples, the processor of either one of clauses 131 or 132 is configured to receive the second signal.

Clause 134: In some examples, the image on at least a portion of the facial shield of any one of clauses 109-133 provides a mixed reality to the user.

Clause 135: In some examples, the projector of any one of clauses 109-134 is configured to control the image on the at least a portion of the facial shield such that it visually corresponds with the implant.

Clause 136: In some examples, the projector of clause 135 is configured to control the image on the at least a portion of the facial shield such that it at least partially overlays the implant.

Clause 137: In some examples, the image of either one of clauses 135 or 136 includes the identity characteristic of the implant.

Clause 138: In some examples, the processor of any one of clauses 109-137 is configured to verify the presence of a plurality of implants within the medical procedure area.

Clause 139: In some examples, the processor of clause 138 is configured to allow the user to make a selection related to all of the plurality of implants.

Clause 140: In some examples, the processor of either one of clauses 138 or 139 is further configured to verify the presence of a plurality of instruments configured to be used with the implants within the medical procedure area.

Clause 141: In some examples, the processor of clause 140 is configured to allow the user to make a selection related to one or more of the plurality of instruments.

Clause 142: In some examples, the system of any one of clauses 109-141 further includes a memory including a database, wherein the processor is configured to make a comparison between the identity characteristic of the implant and the database.

Clause 143: In some examples, the database of clause 142 includes instructions related to the assembly of the implant.

Clause 144: In some examples, the database of either one of clauses 142 or 143 includes instructions related to use of the implant.

Clause 145: In some examples, the database of any one of clauses 142-144 includes instructions related to implantation of the implant.

Clause 146: In some examples, the database of any one of clauses 142-145 includes guidelines for proper implantation of the implant.

Clause 147: In some examples, the database of any one of clauses 142-146 includes a list of options of configurations utilizing the implant.

Clause 148: In some examples, the system of either one of clauses 140 of 141 further includes a memory including a database, wherein the processor is configured to make a comparison between an identity characteristic of at least one of the instruments and the database.

While the foregoing is directed to embodiments of the present disclosure, other and further embodiments may be devised without departing from the basic scope thereof.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

1. A system for improving efficiency in a medical facility comprising:
one or more sensors configured to wirelessly obtain information from a medical procedure area and output a first signal related to the obtained information;
a processor configured to survey the first signal and identify one or more descriptors in the signal; and
an output configured to describe one or more changes related to operation of the medical procedure area based at least in part on the one or more descriptors.

2-12. (canceled)

13. The system of claim 1, wherein the one or more sensors is configured to identify the location of a person.

14. The system of claim 1, wherein the processor is configured to aggregate the obtained information with one or more groups of saved information.

15. The system of claim 14, wherein the processor is configured to make comparisons between the obtained information and at least one of the one or more groups of saved information.

16. The system of claim 15, wherein the processor comprises a clock, and wherein the comparisons utilize the clock.

17. The system of claim 1, further comprising a remote device containing an application configured to communicate with the processor.

18. The system of claim 15, wherein the obtained information comprises one or more voice conversations.

19-31. (canceled)

32. The system of claim 1, wherein the processor is configured to cross-reference a database including stored data related to a medical procedure.

33. The system of claim 32, wherein the processor comprises an artificial intelligence (AI) apparatus.

34. The system of claim 32, wherein the processor is configured to compare the stored data with a characteristic from the list consisting of: time, efficiency, and procedural outcome.

35-41. (canceled)

42. A system for aiding a medical procedure comprising:
a cover configured to protect the head of a user from contaminants in an ambient environment external to the cover, the cover comprising a substantially transparent facial shield;
a projector configured to project an image on at least a portion of the facial shield that is configured to reside within a field of vision of the user;
a sensor configured to wirelessly obtain information from a medical implant within a medical procedure area and output a first signal related to the obtained information; and
a processor configured to determine an identity characteristic of the implant based on the first signal.

43. The system of claim 42, wherein the processor is configured to determine the identity characteristic of the implant without requiring the receipt of any verbal command.

44. (canceled)

45. The system of claim 42, wherein the processor is configured to receive non-verbal information from the user.

46. The system of claim 45, wherein the non-verbal information is selected from the list consisting of: head movement, eyelash movement, eyelid movement, eye movement, nose movement, facial skin movement, mandible movement, ear movement, tongue movement, lip movement, breath flow, mouth heat, breath heat.

47. The system of claim 45, wherein the non-verbal information comprises one or more non-verbal mouth noise.

48. The system of claim 47, wherein the one or more non-verbal mouth noise comprises a noise selected from the list consisting of: a click, a pop, a puff, a hiss, a sniff, a cough, a sibilance, a whistle, and a gurgle.

49-57. (canceled)

58. The system of claim 42, wherein the processor is configured to verify the presence of a plurality of implants within the medical procedure area.

59. (canceled)

60. The system of claim 58, wherein the processor is further configured to verify the presence of a plurality of instruments configured to be used with the implants within the medical procedure area.

61. (canceled)

62. The system of claim 42, further comprising a memory comprising a database, wherein the processor is configured to make a comparison between the identity characteristic of the implant and the database.

63-73. (canceled)

74. The system of claim 42, wherein the cover comprises at least one of a personal protection feature selected from the list consisting of: a helmet, a hood, a shroud, a bonnet, and a cuff.
